(19) United States
(12) Patent Application Publication
(10) Pub. No.: US 2003/0050293 A1
(43) Pub. Date: Mar. 13, 2003

(54) Use of dehydroepiandrosterone, its precursors and derivatives, by oral administration, as hydrating agents with direct action

(76) Inventors: Olivier De La Charriere, Paris (FR); Stephanie Nouveau, Boulogne Billancourt (FR); Francoise Forette, Paris (FR); Etienne Emile Baulieu, Neuilly Sur Seine (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/181,002

(22) PCT Filed: Jan. 17, 2001
(86) PCT No.: PCT/FR01/00138

(30) Foreign Application Priority Data
Jan. 17, 2000 (FR) 00/00551

(51) Int. Cl. A61K 31/56; A61K 7/00

(52) U.S. Cl. 514/177; 514/178; 424/401

(57) ABSTRACT

The invention concerns the use of at least a compound selected in the group consisting of dehydroepiandrosterone, its chemical precursors, its chemical derivatives, its biological precursors and metabolic derivatives, for preparing a composition designed for oral administration and for use to directly increase skin water content.

The present invention relates to the use of dehydroepiandrosterone, its precursors and its derivatives, for producing a composition suitable for oral administration and intended to be used to directly increase the water content of the skin.

During aging, a decrease in the endogenous secretion of sebum is observed, both in men and women (P. E. Pochi in Advances in Biology of skin, (1965), vol. VI, Aging, edited by W. Montagna, Pergamon Press, N.Y.), the consequence of which is thought to be drying out of the skin.

In order to combat this decrease in the secretion of sebum, a testosterone-based treatment has been proposed (E. Pochi (1965); reference cited), but the side effects observed in women mean that this treatment is rarely used.

Dehydroepiandrosterone (DHEA) is a natural steroid produced essentially by the adrenocortical glands. It is known for its antiaging properties related to its ability to promote epidermal keratinization (JP-07196467) and to combat osteoporosis (U.S. Pat. No. 5,824,671). It is used in the treatment of obesity and of diabetes (WO 97/13500) and in various diseases of hormonal origin, such as some cancers (WO 94/16709). It has also been proposed to use DHEA sulfate against alopecia (JP-60142908) and for treating the various signs of aging, such as elasticity, flaccidity or slackening of the skin (EP-0723775).

DHEA-based compositions intended to decrease drying out of the skin are described in patent U.S. Pat. No. 4,496,556. In these compositions, which can be administered orally, DHEA induces an increase in the endogenous production of sebum and in its secretion. The increase in the production of sebum forms an occluding layer on the skin, which reinforces the cutaneous barrier and allows water retention in the stratum corneum, thus preventing drying out of the skin.

However, none of these documents either describes or suggests the oral use of DHEA, its precursors and its derivatives, for directly increasing the water content of the skin, which is the subject of the present invention.

Specifically, the applicant has found, unexpectedly, that DHEA, or at least one of its chemical precursors, of its chemical derivatives, of its biological precursors or of its metabolic derivatives, administered orally, is capable of acting directly on the water content of the skin, this being independently of their action on seborrhea.

Consequently, the subject of the present invention is the use of at least one compound chosen from the group consisting of dehydroepiandrosterone, its chemical precursors, its chemical derivatives, its biological precursors and its metabolic derivatives, for preparing a composition suitable for oral administration and intended to be used to directly increase the water content of the skin.

In an advantageous embodiment of the use according to the invention, the biological precursor is chosen from the group comprising cholesterol, pregnenolone, 17α-hydroxypregnenolone, 5-androstenediol, dehydroepiandrosterone sulfate, 17α-hydroxypregnenolone sulfate and 5-androstenediol sulfate.

In another advantageous embodiment of this use, the chemical precursor is chosen from the group comprising sapogenins, such as, for example, diosgenin (or spirost-5-en-3-β-ol) and the natural extracts containing them, in particular fenugreek and the extracts of Dioscoreaceae such as wild yam.

In another advantageous embodiment of this use, the metabolic derivative is chosen from the group comprising 5-androstene-3β-17β-diol (adiol), 5-androstene-3β-17β-diol sulfate and 4-androstene-3,17-dione.

In another advantageous embodiment of this use, the chemical derivative is chosen from the group comprising the salts of DHEA, in particular the water-soluble salts of DHEA, such as for example DHEA sulfate, and the esters of DHEA, in particular DHEA salicylate.

In accordance with the use according to the invention, the composition may be a cosmetic composition or a dermatological composition, provided in all the pharmaceutical forms normally used for oral administration, in particular in the form of tablets which may or may not be breakable, of granules, of capsules, of gelatin capsules, of solutes, of suspensions or of solutions, and comprising at least one compound chosen from the group consisting of dehydroepiandrosterone, its precursors and its derivatives, as active principle, in combination with any suitable excipient.

In accordance with the use according to the present invention, the compounds are present in the composition in an amount which allows them to be administered at a dose of between 1 and 100 mg per day, preferably between 25 and 75 mg per day, said dosage being taken in one or more stages, for example with a unit dose of 50 mg.

The DHEA which can be used according to the invention is, for example, available from the company SIGMA or from the company AKZO NOBEL.

Other characteristics and advantages of the invention appear in the remainder of the description and the examples.

### EXAMPLE 1

### Evaluation of the Direct Effect of DHEA Administered Orally on the Water Content of the Skin and of its Effect on the Production of Sebum—Comparison with a Placebo

1. Methodology

1.1. Treatment

The evaluation was carried out using a randomized double-blind clinical study among 280 individuals (140 men and 140 women). The inclusion criteria was as follows: healthy volunteers, of both sexes, free of any severe pathological condition and 60 to 80 years old.

The treatment consisted, for 12 months, of a daily dose of 50 mg of DHEA, taken in the morning, in the form of a breakable tablet, for half of the individuals, or of a placebo for the other half of the individuals.

1.2. Evaluation of the Moisturization of the Skin

The moisturization of the skin on the ventral side of the forearm is measured, blind, at the start of treatment (T0) and at the end of treatment (T12), by the same clinician, using a Dermodiag®, which is a device described in patent U.S. Pat. No. 5,001,436. The characteristics of the device are as follows: outlet impedance: 1.4 kOhms and working frequency 10 MHz. The method used is that described by J. L. Lévêque, Soc. Cosmet. Chem. (1983), 34, 419-428.

This device makes it possible to evaluate the state of moisturization (water content of the superficial layers of the epidermis) by measuring electrical conductance. The conductance is measured using an electrode applied to the skin for 10 seconds. The conductance value is the result of the mean of 6 individual measurements per measurement time.

The statistical analysis is performed using the Wilcoxon test.

1.3. Evaluation of the Production of Sebum

The production of sebum is measured at the start of treatment (T0) and at the end of treatment (T12) according to the techniques described by A. M. Kligman et al., J. Soc. Cosmet. Chem. (1986), 37, 369-373 and by K. M. Nordstrom et al., J. Invest. Dermatol. (1986), 87, 260-264.

White adhesive microporous patches (Sebutape®, Cuderm Corporation, Dallas, Tex., USA) described in U.S. Pat. No. 4,532,937 are used; the patches are applied in a standardized manner to the skin, which has been wiped 10 times, beforehand, with cotton soaked in 70° alcohol in order to remove any fat. The lipids which are passively absorbed onto the patches are visualized by the presence of transparent spots, themselves measured by image analysis.

Each spot represents an active sebaceous gland.

The statistical analysis is performed using the Wilcoxon test.

2. Results:

They are illustrated in tables 1 and 2.

TABLE 1

| | | | | | Significance |
|---|---|---|---|---|---|
| | DHEA | | Placebo | | (Wilcoxon |
| Conductance | T0 | T12 | T0 | T12 | test) |
| Women | 64 | 84 | 58 | 79 | P = 0.44 |
| Men | 69 | 86 | 68 | 82 | P = 0.03 |

TABLE 2

| | | | | | Significance |
|---|---|---|---|---|---|
| Number of | DHEA | | Placebo | | (Wilcoxon |
| spots | T0 | T12 | T0 | T12 | test) |
| Women | 2 | 56 | 2 | 19 | P = 0.0001 |
| Men | 55 | 140 | 31 | 131 | P = 0.46 |

Among the men, the oral treatment with DHEA allows significant improvement (p=0.03) of the moisturization of the skin, measured by conductance (+26% in the treated group against +21% in the placebo group), whereas no significant variation in seborrhea is observed after treatment with DHEA (p=0.46).

On the other hand, among the women, the treatment with DHEA leads to a significant variation in seborrhea (p=0.0001) without significant variation in the moisturization of the skin (p=0.44).

These results show that DHEA, administered orally, has a direct effect on the water content of the skin and that this effect is not related to seborrhea.

1. The use of at least one compound chosen from the group consisting of dehydroepiandrosterone, its chemical precursors, its chemical derivatives, its biological precursors and its metabolic derivatives, for preparing a composition suitable for oral administration and intended to be used to directly increase the water content of the skin.

2. The use as claimed in claim 1, characterized in that the biological precursor is chosen from the group comprising cholesterol, pregnenolone, 17α-hydroxypregnenolone, 5-androstenediol, dehydroepiandrosterone sulfate, 17α-hydroxypregnenolone sulfate and 5-androstenediol sulfate.

3. The use as claimed in claim 1, characterized in that the chemical precursor is chosen from the group consisting of sapogenins and the natural extracts containing them.

4. The use as claimed in claim 1, characterized in that the metabolic derivative is chosen from the group comprising 5-androstene-3β-17β-diol (adiol), 5-androstene-3β-17β-diol sulfate and 4-androstene-3,17-dione.

5. The use as claimed in claim 1, characterized in that the chemical derivative is chosen from the group consisting of the salts and the esters of DHEA.

6. The use as claimed in any one of claims 1 to 5, characterized in that the composition is a cosmetic composition.

7. The use as claimed in any one of claims 1 to 5, characterized in that the composition is a dermatological composition.

8. The use as claimed in any one of claims 1 to 7, characterized in that the compounds are present in the composition in an amount which allows them to be administered at a dose of between 1 and 100 mg per day, preferably between 25 and 75 mg per day.
